(19) United States
(12) Patent Application Publication
(10) Pub. No.: US 2011/0034510 A1
(43) Pub. Date: Feb. 10, 2011

(54) ENANTIOMER-ENRICHED ALPHA-,OMEGA-AMINO ALCOHOL DERIVATIVES, THEIR PRODUCTION AND USE AS INSECT- AND MITE-REPELLING AGENTS

(75) Inventors: Thomas Gernot, Hurth (DE); Nikolaus Mueller, Wallhausen (DE); Andreas Job, Cologne (DE); Bernd-Wieland Kruger, Bergisch Gladbach (DE); Gunther Nentwig, Leverkusen (DE)

Correspondence Address: LANXESS CORPORATION, 111 RIDC PARK WEST DRIVE, PITTSBURGH, PA, 15275-1112, US

(73) Assignee: SALTIGO GMBH, Langenfeld (DE)

(21) Appl. No.: 12/445,622

(22) PCT Filed: Oct. 18, 2007
(86) PCT No.: PCT/EP2007/061164

§ 371 Date: Oct. 29, 2010

(30) Foreign Application Priority Data
Oct. 21, 2006 (DE) 10 2006 049 763.5

(51) Int. Cl. A01N 43/40; C07D 211/22; A01P 17/00

(52) U.S. Cl. 514/315; 546/245

(57) ABSTRACT

The invention relates to an insect- and mite-repelling agent, characterized by a content of at least one substituted, enantiomer-enriched α-,ω-amino alcohol derivative of formula (1) wherein X represents hydrogen, COR¹¹ or R¹³, R¹³ represents C₁-C₆ alkyl, R¹ represents C₁-C₇ alkyl, C₃-C₇alkenyl or C₂-C₇ alkinyl, R² R¹¹, R¹³ are identical or different and represent C₁-C₆ alkyl or C₂-C₇ alkenyl, R³-R⁸ are identical or different and represent hydrogen or C₁-C₆ alkyl, wherein R² and R³ or R³ and R⁷ or R³ and R⁵ or R⁵ and R⁷ can also form, together with the atoms to which they are bonded, a 5- or 6-membered monocycle ring. Also disclosed is a method for producing said agent.

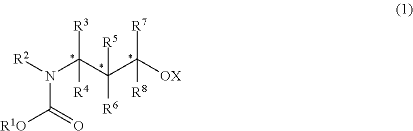

The present invention relates to the preparation and use of enantiomerically enriched substituted α,ω-amino alcohol derivatives as insect and mite repellents. The present invention further provides novel substituted α,ω-amino alcohol derivatives.

Insect or mite repellents have the task of preventing harmful or annoying arthropods from touching, and also from stinging and sucking or biting, on surfaces which attract them, for instance on the skin of animals and humans when they have been treated beforehand with such repellents.

Numerous active ingredients have already been proposed as repellents (cf., for example, K. H. Büchel in Chemie der Pflanzenschutz- und Schädlingsbekämpfungsmittel; [Chemistry of Crop Protectants and Pesticides]; editor: R. Wegler, vol. 1, Springer Verlag Berlin, Heidelberg, New York, 1970, p. 487 ff.).

Particularly well known examples which have been in use for sometime are N,N-diethyl-3-methylbenzamide (DEET), dimethyl phthalate and 2-ethylhexane-1,3-diol, of which DEET in particular has gained considerable significance in practice (see, for example, R. K. Kocker, R. S. Dixit, C. I. Somaya; Indian J. Med. Res. 62,1 (1974)).

A considerable disadvantage of the known repellents is their sometimes relatively short-lived duration of action (only a few hours).

Some of the compounds defined by the formula (1) which follows are known in the form of racemates thereof (on this subject, see DE 1 288 587, column 2, formula 1 with the definition of R=methyl). This is an intermediate for the preparation of ethyl N-(3-carbamoyloxyalkyl)carbamate, which is used as a sedative medicament (on this subject, see EP 0 144 825 A1, compound No. 37 on page 43), which serves as an intermediate for the preparation of antibiotic compounds.

DE-A 1 150 973 likewise discloses some compounds of analogous structure, which are used as medicaments.

However, no insect- and mite-repellent action of these compounds has become known to date.

Substituted α,ω-amino alcohol derivatives of the formula (1) are also known in the form of their racemic mixture of the individual enantiomers, as obtained in the chemical synthesis (EP A 289 842),

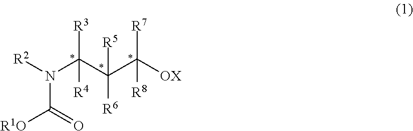

in which
X is hydrogen, COR¹¹ or R¹³, where R¹³ is C₁-C₆-alkyl,
R¹ is branched or linear C₁-C₇-alkyl, C₃-C₇-alkenyl or C₂-C₇-alkynyl,
R² is C₁-C₆-alkyl or C₂-C₇-alkenyl,
R³ to R⁸ are the same or different and are each hydrogen or C₁-C₆-alkyl, where R² and R³ or R³ and R⁷ or R³ and R⁵ or R⁵ and R⁷, together with the atoms to which they are bonded, may also form a 5- or 6-membered monocyclic ring.

It was an object of the invention to provide a more effective insect and mite repellent.

It has now been found that the individual optical isomers of these amino alcohols of the formula 1 have different action as insect and mite repellents.

Even in the form of the racemic isomer mixtures used, these exhibit a strong insect- and mite-repellent action.

As in the case of all substances which occur in optical isomers, the biological action of the individual enantiomers may be quite different, such that it appears to be advantageous to prepare the individual optical isomers and to check their biological activity.

The most effective isomer can then be prepared in enriched or enantiomerically enriched faun and be marketed as a more effective insect and mite repellent. The advantage of this procedure is a significant reduction in the amount employed and “omission” of the less effective or ineffective isomers. In addition to an increase in the efficiency over the enantiomer mixtures, this prevents ineffective isomers burdened with possibly greater undesired side effects from being employed.

The invention therefore provides an insect and mite repellent, characterized by a content of at least one substituted enantiomerically enriched α,ω-amino alcohol derivative of the formula (1)

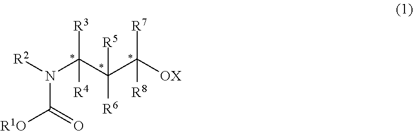

in which
X is hydrogen, COR¹¹ or R¹³, where R¹³ is C₁-C₆-alkyl,
R¹ is C₁-C₇-alkyl, C₃-C₇-alkenyl or C₂-C₇-alkynyl,
R², R¹¹, R¹³ are the same or different and are each C₁-C₆-alkyl or C₂-C₇-alkenyl,
R³ to R⁸ are the same or different and are each hydrogen or C₁-C₆-alkyl, where R² and R³ or R³ and R⁷ or R³ and R⁵ or R⁵ and R⁷, together with the atoms to which they are bonded, may also form a 5- or 6-membered monocyclic ring.

The compounds of the general formula (1) can be prepared in analogy to the racemic mixtures by known methods and processes (cf., for example, C. Ferri, Reaktionen der organischen Synthese [Reactions in Organic Synthesis], Georg Thieme Verlag Stuttgart, 1978, p. 211 ff. and 496, 497).

Accordingly, the compounds of the formula (1) are obtained when the optically active α,ω-amino alcohols preparable by known processes (e.g. (S)- or (R)-2-(2-hydroxyethyl)piperidine→S. M. Kupchun et al. J. Am. Chem. Soc. 82 (1960) 2616) of the formula (2)

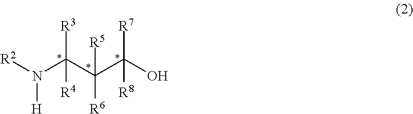

in which
R² to R⁸ are each defined as specified in formula (1)

are first reacted with chlorocarbonic esters, known per se, of the formula (3) in which R¹ is an optically active radical, for example (R)- or (S)-sec-butyl

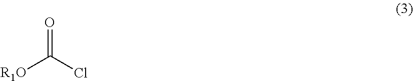

and is otherwise defined as specified in the formula (1), optionally in the presence of an acid acceptor, for example triethylamine or potassium carbonate, and optionally using a diluent, for example toluene, CH₂Cl₂, tetrahydrofuran or acetonitrile, at temperatures between −40 and 110° C.

In the case that R² and R³ together with the atoms to which they are bonded can form a 6-membered ring (piperidine), the chiral amino alcohol of the formula (2) can be prepared by a novel method by chiral ring hydrogenation of a corresponding pyridine (WO 2005/049570).

For the preparation of compounds of the general formula (1) in which X is other than hydrogen, in a second reaction step, optionally after isolation of the intermediate with a free OH group, the further acylation/alkylation is effected with carbonyl chlorides, known per se, of the formula (4)

R¹¹COCl (4)

to prepare compounds of the formula (1) where X═COR¹¹;

or alkyl halides of the formula (6)

R¹³—Y (6)

to prepare compounds of the formula (1) where X═R¹³;

where, in the formulae (4), (6), Y is chlorine, bromine or iodine, preferably bromine or iodine, and R¹¹ and R¹³ are each as defined above,

optionally in the presence of an acid acceptor, for example triethylamine or potassium carbonate, or of a base such as sodium hydride or butyllithium, optionally using a diluent, for example toluene, tetrahydrofuran or acetonitrile, at temperatures between −78 and 110° C.,

b) the compounds of the formula (1) are also obtained when the α,ω-amino alcohols or α,ω-amino ethers, known per se or preparable by known processes, of the formula (10)

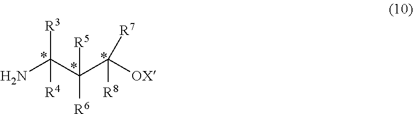

in which

R³ to R⁸ are each defined as specified in formula (1) and where X′ is hydrogen or R¹³, are first reacted with chlorocarbonic esters, known per se, with an optically active alkyl radical R¹ of the formula (3), optionally in the presence of an acid acceptor, for example triethylamine or potassium carbonate, or optionally using a diluent, for example toluene, CH₂Cl₂, tetrahydrofuran or acetonitrile, at temperatures between −40 and 110° C.

In a second reaction step, for the preparation of compounds of the formula (1) in which X is not R¹³ or hydrogen, optionally after isolation of the intermediate with a free OH group, the further acylation is then carried out with carbonyl chlorides, known per se, of the formula (4) to prepare compounds of the formula (1) where X═COR¹¹, where R¹¹ in the formula (4) is defined as specified above, optionally in the presence of an acid acceptor, for example triethylamine or potassium carbonate, or optionally using a diluent, for example toluene, tetrahydrofuran or acetonitrile, at temperatures between −78 and 110° C. In a third reaction step, optionally after isolation of the intermediate with a free NH group, the further N-alkylation is then carried out with alkyl halides of the formula (11)

R²—Y′ (11)

to prepare compounds of the formula (1) in which Y′ is chlorine, bromine or iodine, preferably bromine or iodine, and R² is defined as specified above, optionally in the presence of a base, for example sodium hydride or butyllithium, optionally using a diluent, for example toluene or tetrahydrofuran, at temperatures between −78 and 110° C.

The workup is effected by customary methods, for example by extracting the products with methylene chloride or toluene from the water-diluted reaction mixture, washing the organic phase with water, drying and distilling, or so-called “incipient distillation”, i.e. by prolonged heating under reduced pressure to moderately elevated temperatures, in order to free the product of the last volatile constituents.

A further purification can be effected by chromatography on silica gel with, for example, hexane:acetone=7:3 as the eluent. The compounds are characterized using NMR spectrum, refractive index, melting point, Rf or boiling point. The optical purity is determined by known methods, for example NMR with addition of chiral shift reagents, or by gas chromatography on columns with chiral carrier material.

The present invention also relates to novel substituted optically active amino alcohol derivatives of the formula (7)

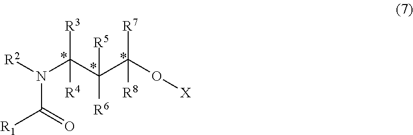

in which
X is hydrogen, COR¹¹ or R¹³, where R¹³ is C₁-C₆-alkyl,
R¹ is C₁-C₇-alkyl, C₃-C₇-alkenyl or C₂-C₇-alkynyl,
R², R¹¹, R¹³ are the same or different and are each C₁-C₆-alkyl or C₂-C₇-alkenyl,
R³-R⁸ are the same or different and are each hydrogen or C₁-C₆-alkyl, where R² and R³ or R³ and R⁷ or R³ and R⁵ or R⁵ and R⁷, likewise together with the atoms to which they are bonded, may also form a 5- or 6-membered monocyclic ring.

This excludes the following substituent combinations a) to f):
a) X=hydrogen, R²=methyl and R¹=tert-butyl
b) X=hydrogen, R¹=ethyl, R⁵=ethyl, R⁶=ethyl
c) X, R³, R⁴, R⁷, R⁸=hydrogen, R¹, R⁵, R⁶=ethyl, R²=methyl
d) X, R³, R⁴, R⁸=hydrogen, R¹, R², R⁵, R⁶=ethyl, R⁷=methyl
e) X, R³, R⁴, R⁷, R⁸=hydrogen, R¹, R², R⁵, R⁶=ethyl
f) X, R³, R⁴, R⁷, R⁸=hydrogen, R¹, R⁵, R⁶=ethyl, R²=n-propyl.

The compounds of the formula (7) are obtained
a) when the chiral α,ω-amino alcohols, known per se or preparable by known processes (cf., for example, B. Cesare Ferri, Reaktionen der org. Synthese, Georg Thieme Verlag Stuttgart, 1978, p. 211 ff. or 496-497), of the formula

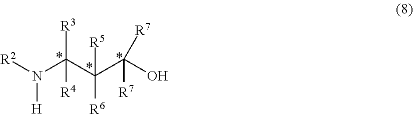

in which

R² to R⁸ are each defined as specified in formula (7) are first reacted with carbonic acid derivatives, known per se, of the formula (9)

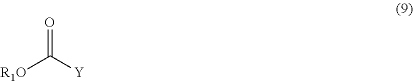

where R¹ is defined as specified in formula 7 and Y is halogen or a leaving group customary in amidation reactions, preferably an activating ester radical or a group

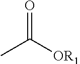

optionally in the presence of a diluent and optionally with addition of a base.

For the preparation of compounds of the general formula (7) in which X is other than hydrogen, in a second reaction step, optionally after isolation of the intermediate with a free OH group, the further acylation/alkylation is then effected with carbonyl chlorides, known per se, of the formula (4)

R¹¹COCl (4)

to prepare compounds of the formula (7) where X═COR¹¹, or alkyl halides of the formula (6)

R¹³—Y (6)

to prepare compounds of the formula (1) where X═R¹³, where Y in the formulae (4), (6) is chlorine, bromine or iodine, preferably bromine or iodine, and R¹¹ and R¹³ are each as defined above, the reaction optionally being effected in the presence of an acid acceptor, for example triethylamine or potassium carbonate, or of a base such as sodium hydride or butyllithium, optionally using a diluent, for example toluene, tetrahydrofuran or acetonitrile, at temperatures between −78 and 110° C.
b) The compounds of the formula (7) are also obtained when the chiral α,ω-amino alcohols or α,ω-amino ethers, known per se or preparable by known processes, of the formula (12)

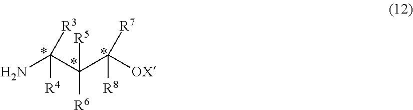

in which

R³ to R⁸ are each defined as specified in formula (7) and where X′ is hydrogen or R¹³, where R¹³ is optionally substituted alkyl or alkenyl, are first reacted with chiral chlorocarbonic esters, known per se, of the formula (3), optionally in the presence of an acid acceptor, for example triethylamine or potassium carbonate, and optionally using a diluent, for example toluene, CH₂Cl₂, tetrahydrofuran or acetonitrile, preferably at temperatures between −40 and 110° C.

In a second reaction step, for the preparation of compounds of the formula (7) in which X is not R¹³ or hydrogen, optionally after isolation of the intermediate with a free OH group, the further acylation is effected with carbonyl chlorides, known per se, of the formula (4) to prepare compounds of the formula (7) where X═COR¹¹, where R¹¹ and R¹² in the formula (4) are each defined as specified above, optionally in the presence of an acid acceptor, for example triethylamine or potassium carbonate, optionally using a diluent, for example toluene, tetrahydrofuran or acetonitrile, at temperatures between −78 and 110° C.

In a third reaction, optionally after isolation of the intermediate with a free NH group, the further N-alkylation is then effected with alkyl halides of the formula (11)

R²—Y′ (11)

to prepare compounds of the formula (7) where Y′ is chlorine, bromine or iodine, preferably bromine or iodine, and R² is defined as specified above, optionally in the presence of a base, for example sodium hydride or butyllithium, optionally using a diluent, for example toluene or tetrahydrofuran, at temperatures between −78 and 110° C.

The workup is effected by customary methods, for example by extraction of the products with methylene chloride or toluene from the water-diluted reaction mixture, washing of the organic phase with water, drying and distilling, or so-called “incipient distillation”, i.e. by prolonged heating under reduced pressure to moderately elevated temperatures, in order to free it from the last volatile constituents.

Further purification can be effected by chromatography on silica gel with, for example, hexane:acetone=7:3 as the eluent.

The novel substituted optically active α,ω-amino alcohol derivatives of the general formula (7) are notable for strong insect- and mite-repellent action. They can also be used in synergistic mixtures with other repellents.

The radicals specified in the formula (7) are preferably each defined as follows:

Examples of the alkyl group in the R¹ to R¹³ radicals include: methyl, ethyl, n- and i-propyl, n-, s- and t-butyl, n-pentyl and n-hexyl.

Examples of alkenyl include: 2-propenyl, 2-butenyl and 3-butynyl.

Particular preference is given to compounds of the general formula (7) in which
X is hydrogen or R¹³, where R¹³ is C₁-C₆-alkyl,
R¹ is C₁-C₇-alkyl or C₃-C₇-alkenyl,
R⁴ to R⁸ are the same or different and are each hydrogen or C₁-C₆-alkyl,
R² and R³ together with the atoms to which they are bonded, may also form a 5- or 6-membered monocyclic ring.

Additionally preferred are compounds of the formula (7) in which R¹ is C₁-C₇-alkyl, C₃-C₇-alkenyl or C₂-C₇-alkynyl, X is hydrogen, COR¹¹ or R¹³, R² and R¹¹ are the same or different and are each C₁-C₆-alkyl, R³ to R⁸ are the same or different and are each hydrogen or C₁-C₆-alkyl, R¹³ is C₁-C₆-alkyl,

excluding the following substituent combinations a) to f):
a) X=hydrogen, R²=methyl and R′=tert-butyl,
b) X=hydrogen, R′=ethyl, R⁵=ethyl, R⁶=ethyl
c) X, R³, R⁴, R⁷, R⁸=hydrogen, R¹, R⁵, R⁶=ethyl, R²=methyl
d) X, R³, R⁴, R⁸=hydrogen, R¹, R², R⁵, R⁶=ethyl, R⁷=methyl
e) X, R³, R⁴, R⁷, R⁸=hydrogen, R¹, R², R⁵, R⁶=ethyl
f) X, R³, R⁴, R⁷, R⁸=hydrogen, R¹, R⁵, R⁶=ethyl, R²=n-propyl.

When, for example, S-(+)-2-(2-hydroxyethyl)piperidine and sec-butyl (R)-(−)-chloroformate are used as starting materials, the reaction of these compounds can be outlined by the following formula scheme:

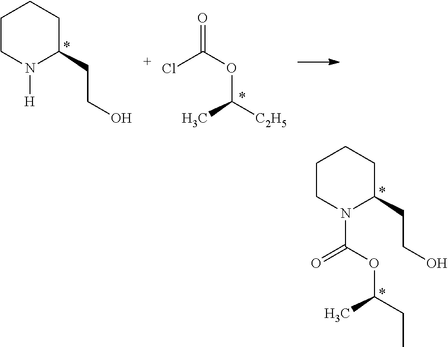

Particular preference is given to enantiomerically enriched substituted α,ω-amino alcohol derivatives of the formula (7), characterized in that they are from the group of 1-[(S)-sec-butyloxycarbonyl]-2-(S)-(2-hydroxyethyl)piperidine, 1-[(R)-sec-butyloxycarbonyl]-2-(R)-(+)-(2-hydroxyethyl)piperidine, 1-[(S)-sec-butoxycarbonyl]-2-(R)-(+)-(2-hydroxyethyl)piperidine or 1-[(R)-sec-butyloxycarbonyl)-2-(S)-(+)-(2-hydroxyethyl)piperidine. Particular preference is given to 1-[(R)-sec-butyloxycarbonyl)-2-(S)-(2-hydroxyethyl)piperidine and 1-[(R)-sec-butyloxycarbonyl)-2-(R)-(2-hydroxyethyl)piperidine.

The action of the repellents of the general formula (1 or 7) is long-lasting. They can therefore be used with good success for repellency of harmful or annoying, sucking and biting insects and mites.

The sucking insects include essentially the mosquitoes (e.g. Aedes, Culex and Anopheles species), sandflies (Phlebotoma), biting midges (Culicoides species), blackflies (Simulium species), biting houseflies (e.g. Stomoxys calcitrans), tsetse flies (Glossina species), horseflies (Tabanus, Haematopota and Chrysops species), common houseflies (e.g. Muca domestica and Fannia canicularis), fleshflies (e.g. Sarcophaga carnaria), flies which cause myiasis (e.g. Lucilia couprina, Chrysomyia chloropyga, Hypoderma bovis, Hypoderma lineatum, Dermatobia hominis, Oestrus ovis, Gasterophilus intestinalis, Cochliomyia hominovorax), bugs (e.g. Cimex lectularius, Rhodnius prolixus, Triatoma infestans), lice (e.g. Pediculus humanus, Haematipinus suis, Damalina ovis), louse flies (e.g. Melaphagus orinus), fleas (e.g. Pulex irritans, Cthenocephalides canis, Xenopsylla cheopsis) and sandfleas (e.g. Dermatophilus penetrans).

The biting insects include essentially cockroaches (e.g. Blattela germanica, Periplaneta americana, Blatta orientalis, Supella supellectilium), beetles (e.g. Sitophilus granarius, Tenebrio molitor, Dermestes lardarius, Stegobium paniceum, Anobium punctactum, Hylotrupes bajulus), termites (e.g. Reticulitermes lucifugus) and ants (e.g. Lasius niger).

The mites include ticks (e.g. Ornithodorus Moubata, Ixodes ricinus, Boophilus microplus, Amblyomma hebreum), and mites in the narrower sense (e.g. Sarcoptes scabiei, Dermanyssus gallinae).

The present invention thus relates to the preparation and use of optically active substituted α,ω-amino alcohol derivatives of the general formula 1 for insect and mite repellency.

The invention further relates to insect and mite repellents, characterized by the content of at least one substituted α,ω-amino alcohol derivative of the general formula (1) or (7).

The inventive repellents which comprise at least one derivative of the formula (1) or (7) may also comprise further insect repellents. All repellents customary in practice are useful here (cf., for example, K. H. Büchel in Chemie der Pflanzenschutz- und Schädlingsbekämpfungsmittel; editor: R. Wegler, vol. 1, Springer Verlag Berlin, Heidelberg, New York, 1970, p. 487 ff.).

In the case of repellent combinations, preference is given to using the substituted α,ω-amino alcohol derivatives of the general formula 1 together with repellent carboxamides, 1,3-alkanediols and carboxylic esters. Specific examples include: N,N-diethyl-3-methylbenzamide (DEET), 2-ethylhexane-1,3-diol (Rutgers 612) and dimethyl phthalate.

The substituted α,ω-amino alcohol derivatives usable in accordance with the invention are characterized by the general formula (1).

Preference is given to using enantiomerically enriched compounds of the general formula (1) as repellents, in which
X is hydrogen or R¹³, where R¹³ is C₁-C₆-alkyl,
R¹ is linear or branched C₁-C₇-alkyl or C₃-C₇-alkenyl,
R⁴-R⁸ are the same or different and are each hydrogen or C₁-C₆-alkyl,
R² and R³ together with the atoms to which they are bonded, form a 5- or 6-membered monocyclic ring.

Preference is further given to compounds in which R¹ is C₁-C₇-alkyl or C₃-C₇-alkenyl, X is COR¹¹ or R¹³, R² and R¹¹ are the same or different and are each C₁-C₆-alkyl, R³ to R⁸ are the same or different and are each hydrogen or C₁-C₆-alkyl, and R¹³ is C₁-C₆-alkyl.

More particularly, the repellents used are the compounds of the general formula (1) in which R¹ is C₁-C₄-alkyl, R², R¹¹ and R¹³ are the same or different and are each C₁-C₆-alkyl, R³-R⁸ are hydrogens and X is hydrogen, COR¹¹ or R¹³, where R¹¹ and R¹³ are each as defined above.

The repellents used are additionally most preferably compounds of the general formula (1) in which R¹ is C₃-C₄-alkyl, R² and R³ together with the atoms to which they are bonded form a 6-membered ring, R⁴ to R⁸ are each hydrogen and X is hydrogen or R¹³, where R¹³ is C₁-C₄-alkyl.

The inventive active ingredients, which can be used undiluted or preferably diluted, can be converted to the formulations customary for repellents. They can be used in all administration forms customary in cosmetics, for example in the form of solutions, emulsions, gels, ointments, pastes, creams, powders, sticks, sprays or aerosols from spray cans.

For use in the noncosmetic sector, the active ingredients can be incorporated, for example, into granules, oil sprays or slow-release formulations.

The formulations are produced in a known manner by mixing or diluting the inventive active ingredients with solvents (e.g. xylene, chlorobenzenes, paraffins, methanol, ethanol, isopropanol, water), carriers (e.g. polyoxyethylene fatty acid esters, polyoxyethylene fatty alcohol ethers, alkylsulfonates, arylsulfonates) and dispersants (e.g. lignin, sulfite waste liquors, methylcellulose).

The inventive active ingredients can be used in the formulations mixed with one another or else in mixtures with other known active ingredients (e.g. sunscreens). The formulations contain generally between 0.1 and 95% by weight of active ingredient, preferably between 0.5 and 90% by weight.

For protection against blood-sucking insects or ticks, the inventive active ingredients are either applied to the human or animal skin, or items of clothing or other objects are treated with them. The inventive active ingredients are also suitable as an additive of impregnating agents for, for example, textile webs, items of clothing, packaging materials, and as an additive to polishes, cleaners and window cleaners.

The examples of the formulations and the use of the inventive active ingredients which follow serve to further illustrate the invention.

### EXAMPLES

### A) Formulation Examples

### Formulation Example 1

A repellent in the form of a lotion for application to the skin is produced by mixing parts of an inventive active ingredient, 1.5 parts of perfume and 78.5 parts of isopropanol. Isopropanol can be replaced by ethanol.

### Formulation Example 2

A repellent in the form of an aerosol for spraying onto the skin is produced by formulating 50% active ingredient solution consisting of 30 parts of one of the inventive active ingredients, 1.5 parts of perfume, 68.5 parts of isopropanol, with 50% Frigen 11/12 (=halogenated hydrocarbon as a propellant gas) as a spray can preparation. Frigen can be replaced by dinitrogen monoxide or butane.

### Formulation Example 3

Another spray can preparation is composed of 40% active ingredient solution, consisting of 20 parts of one of the inventive active ingredients, 1 part of perfume, 79 parts of isopropanol and 60% propane/butane (ratio 15:85).

Individual formulations were produced corresponding to formulation examples 1, 2 and 3 using the following active ingredients: compounds according to preparation examples Nos. 1, 2, 3, 4.

Repellent Tests:

A) Repellent Test on a Guinea Pig

Test Animal: Aedes aegypti (Imagines)

Solvent: ethanol (99.8%)

3 parts by weight of active ingredient are taken up in 100 parts by volume of solvent

A guinea pig whose back has been shaved in an area of 50 cm² is accommodated in a box such that only the shaved area is accessible to the mosquitoes. After treating the area with 0.4 ml of active ingredient solution, the guinea pig, after evaporation of the solvent, is placed in its box into a cage measuring 60×60×60 cm, which contains mosquitoes of both genders fed only with sugared water.

It is observed for 10 minutes how many mosquitoes bite the guinea pig. Subsequently, it is removed and the test is repeated after 1 hour. The experiment is performed for a maximum of 9 hours or until the effect ceases.

Table A
Repellent test on a guinea pig (Aedes aegypti)

| | | Protection |
|---|---|---|
| Preparation | Formula | time in h |
| 1-[S]-sec- butyloxycarbonyl]-2-(S)- 2-hydroxyethyl)piperidine | 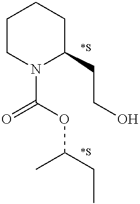 | 3.9 |
| 1-[R]-sec- butyloxycarbonyl]-2-(R)- (2-hydroxyethyl)piperidine | 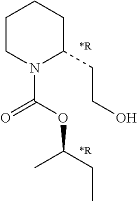 | 4.4 |
| 1-[(R)-sec- butyloxycarbonyl]-2-(S)- (2-hydroxyethyl) piperidine | 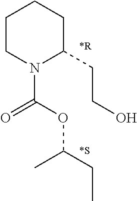 | 3.4 |
| 1-[-(S-sec- butyloxycarbonyl]-2-(R)- 2-hydroxyethyl)piperidine | 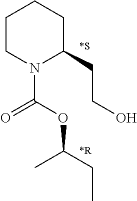 | 4.9 |

B) Repellent Effectiveness of Formulations for Use Against Mosquitoes on the Human Arm:

The insects, as an actively biting population (approx. 1000 mosquitoes of both genders), are kept in cages (length 90 cm, width 30 cm, height 40 cm, side walls made of gauze) which have two light fabric gates on the front side. The insects have been fed exclusively with sugared water (10% Dextropur). The age of the insects is at least 7 days; the number of insects is made up twice per week by 3-day old fully grown insects.

The biting activities are checked every hour continuously during the test period by exposing an untreated arm to the insects (an additional internal product standard is used by a selected volunteer).

The low electrical illumination of the cage is active from 6 am to 6 pm, with light from 6 pm to 6 am. The temperature is 25-27° C.; the relative air humidity is 50-70%.

The lower arms of the test subjects are washed with unperfumed soap, rinsed with water, then rinsed with a solution of 70% ethanol and 30% water, and dried with a towel.

90 cm² of each lower arm of a test subject is rubbed uniformly with 150 μl (or 150 mg) of the test product. As soon as the formulation is dry (after approx. 5 minutes), a sleeve with an opening of 3.1-8 cm (25 cm²) is bound around the arm such that the opening is completely over the treated surface. The corners of the opening of the sleeve have likewise been rubbed with the test material (2000) in a width of 1 cm in order to prevent bites at the corners. The area above the sleeve has been protected with a towel through which the mosquitoes cannot penetrate. Hands are protected with latex gloves.

Both arms are introduced into the cage through the fabric gate, and the number of bites (and landings, if necessary) per arm is noted within a 3-minute test period. The test is repeated every hour for up to 8 hours, or ended beforehand if the effect ceases (3 or more bites within 3 minutes or during 2 successive test sequences). Each test consists of three to 5 test subjects.

Table B reports the increase in the protection time of the inventive enantiomerically enriched compounds compared to the racemic mixture.

Table B
Repellent test against mosquitoes on the human lower arm

| | | Increase in the |
|---|---|---|
| Preparation | Formula | protection time* in % |
| 1-[S]-sec- butyloxycarbonyl]-2-(S)- 2-hydroxyethyl)piperidine | 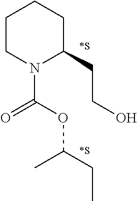 | 0 |
| 1-[(R)-sec- butyloxycarbonyl]-2- (R)-(2- hydroxyethyl)piperidine | 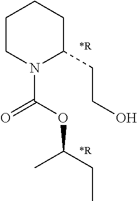 | 3 |
| 1-[(R)-sec- butyloxycarbonyl]-2- (S)-(2- hydroxyethyl)piperidine | 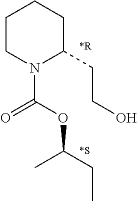 | 12 |
| 1-[(S)-sec- butyloxycarbonyl]-2- (R)-2- hydroxyethyl)piperidine | 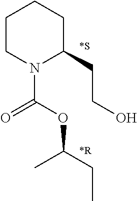 | 0 |

*standard racemic mixture

### B) Preparation Examples

### Example 1

### 1-[(S)-sec-butyloxycarbonyl]-2-(S)-(2-hydroxyethyl)piperidine

### (a) 2-(S)-(2-hydroxyethyl)piperidine camphorsulfonate

A solution of 25.5 g of dl-2-(2-hydroxyethyl)piperidine in 50 ml of ethanol was slowly added dropwise at 0-10° C. to a solution of 64 g of (1S)-(+)-camphor-10-sulfonic acid monohydrate in 80 ml of ethanol. The mixture was left to stand overnight and then 25 ml of ethanol were distilled off. After cooling, precipitation was effected with 600 ml of diethyl ether. After filtration with suction and drying in a drying cabinet, 86 g of almost colorless solid of melting point 125-130° C. (lit. 118-120) were obtained. The solid is then dissolved at 65° C. in 87 ml of ethanol to give an almost clear solution, and hot-filtered. The mother liquor was allowed to come to room temperature overnight and was filtered with suction. The resulting solid, 27 g with a melting point of 137-145° C., was dissolved again in 60 ml of ethanol at 70° C. The mixture was admixed with diethyl ether until it became slightly cloudy and was warmed to room temperature overnight. After the precipitate had been filtered off with suction, 13.7 g of KBR9527-1 with a melting point of 158-160° C. were obtained. Redissolution in 30 ml of ethanol at 70° C. and admixing with diethyl ether until slight cloudiness led, after filtration with suction and drying, to 9 g of d-2-(2-hydroxyethyl)piperidine d-10-camphorsulfonate with a melting point of 168° C. (literature: 168° C., S. M. Kupchan et al. J. Am. Chem. Soc. 82 (1960) 2616).

### (b) Determination of the absolute configuration by x-ray structure analysis

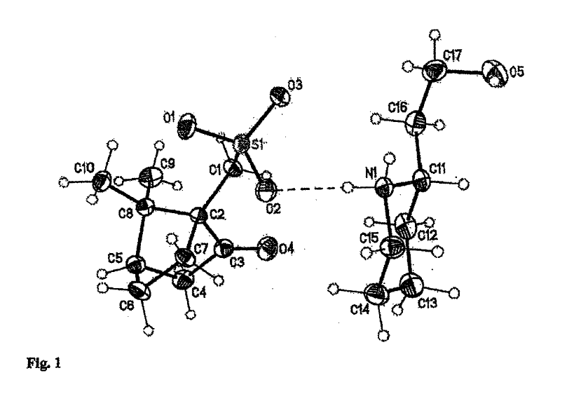

Several crystals of the material obtained above were crystallized by slow evaporation of a saturated acetone solution. A solution with the structure corresponding to proposal (1) was obtained by means of a monoclinic cell using the chiral residual group P2₁. The final configuration is fixed by S(C2); R(C5), R(C11) based on a Flack parameter of −0.03 with a standard deviation of 0.04. Expected measurements were 0 for correct and +1 for mirror-image structures.
The Ortep plot and the exact data are shown in FIG. 1

### (c) 1-[(S)-sec-butyloxycarbonyl]-2-(S)-(2-hydroxyethyl)piperidine

3.6 g (0.0996 mol) of the (+)-camphorsulfonate obtained in 1(a) were initially charged in 50 ml of toluene. At 20° C., 50 ml (0.05 mol) of 1N sodium hydroxide solution were added dropwise with vigorous stirring. After a continued stirring time of 5 minutes, 1.5 g (0.01099 mol) of (S)-(+)-sec-butyl chloroformate are added dropwise at 20° C. Stirring is continued for 1 hour and the organic phase is removed, dried over magnesium sulfate and concentrated fully under reduced pressure.

Yield: 1.95 g (85% of theory) of 1-[(S)-sec-butyloxycarbonyl]-2-(S)-(2-hydroxyethyl)piperidine

[α]_{D}²⁵: −13.2° (CHCl₃)

The other enantiomers were obtained in an analogous manner by the following routes:

1-[(R)-sec-butyloxycarbonyl]-2-(R)-(+)-(2-hydroxyethyl)piperidine

Is from the (−)-camphorsulfonate of 2-(R)-(2-hydroxyethyl)piperidine and R-(−)-sec-butyl chloroformate [α]_{D}²⁵: +12.6° (CHCl₃)

1-[(S)-sec-butyloxycarbonyl]-2-(R)-(+)-(2-hydroxyethyl)piperidine

From the (−)-camphorsulfonate and (R)-(+)-sec-butyl chloroformate

[α]_{D}²⁵: +49.6° (CHCl₃)

1-[(R)-sec-butyloxycarbonyl]-2-(S)-(+)-(2-hydroxyethyl)piperidine

From the (+)-camphorsulfonate and (R)-(−)-sec-butyl chloroformate

[α]_{D}²⁵: −50.6° (CHCl₃)

TABLE 1
Crystal data and structure refining for 2-(S)-
(2-hydroxyethyl)piperidine camphorsulfonate

| | |
|---|---|
| Identification code | Kbr9527 g |
| Empirical formula | C17 H31 N O5 S |
| Formula weight | 361.49 |
| Temperature | 153(2) K |
| Wavelength | 0.71073 Å |
| Crystal system | Monocyclic |
| Space group | P21 |
| Unit cell dimensions | a = 8.804040(10) Å |
| | α = 90° |
| | b = 7.158740(10) Å |
| | β = 92.1410(10)° |
| | c = 14.7546(2) Å |
| | γ = 90° |
| Volume | 929.27(2) Å³ |
| Z | 2 |
| Density (calculated) | 1.292 Mg/m³ |
| Absorption coefficient | 0.200 mm⁻¹ |
| F(000) | 392 |
| Crystal size | 0.30 × 0.30 × 0.30 mm³ |
| Theta range for data collection | 2.31 to 31.40° |
| Index ranges | −12 ≦ h ≦ 12, −10 ≦ |
| | k ≦ 10, −21 ≦ 1 ≦ 21 |
| Reflections collected | 14189 |
| Independent reflections | 5895 [R(int) = 0.0487] |
| Completeness to theta = 31.49° | 96.4% |
| Absorption correction | SADABS (Bruker-AXS) |
| Refinement method | Full-matrix least-squares on F² |
| Data/restraints/parameters | 5895/1/341 |
| Goodness-of-fit on F² | 1.033 |
| Final R indices [I > 2 sigma (I)] | R1 - 0.0382, wR2 = 0.0960 |
| R indices (all data) | R1 = 0.0391, wR2 = 0.0968 |
| Absolute structure parameter | −0.03 (4) |
| Largest diff. peak and hole | 0.255 and −0.343 e · Å⁻³ |

TABLE 2
Bond lengths [Å] and angles [°] for 2-(S)-
(2-hydroxyethyl)piperidine camphorsulfonate

| | | | | | |
|---|---|---|---|---|---|
| S(1)-O(1) | 1.4567(8) | C(2)-O(8) | 1.8649(14) | C(8)-C(9) | 1.5276(15) |
| S(1)-O(2) | 1.4603(9) | C(3)-O(4) | 1.2114(16) | C(11)-C(12) | 1.5223(16) |
| S(1)-O(3) | 1.4660(9) | C(3)-C(4) | 1.5247(17) | C(11)-C(16) | 1.5255(16) |
| S(1)-C(1) | 1.7802(11) | C(4)-C(5) | 1.5385(17) | C(12)-C(13) | 1.532(2) |
| N(1)-C(15) | 1.4962(15) | C(5)-C(6) | 1.5371(16) | C(13)-C(14) | 1.520(2) |
| N(1)-C(11) | 1.5022(13) | C(5)-C(8) | 1.5559(15) | C(14)-C(15) | 1.5214(16) |
| C(1)-C(3) | 1.8272(14) | C(5)-C(17) | 1.4268(18) | C(15)-C(17) | 1.5246(19) |
| C(2)-C(3) | 1.5335(15) | C(6)-C(7) | 1.5501(16) | | |
| C(2)-C(7) | 1.5600(14) | C(8)-C(10) | 1.5239(16) | | |

| | | | |
|---|---|---|---|
| O(6)-S(6)-O(3) | 113.15(6) | C(6)-C(5)-C(8) | 102.28(8) |
| O(1)-S(1)-O(3) | 112.21(5) | C(4)-C(5)-C(8) | 103.09(9) |
| O(2)-S(1)-O(3) | 112.18(6) | C(5)-C(6)-C(2) | 103.36(9) |
| O(1)-S(1)-C(1) | 108.17(6) | C(6)-C(7)-C(2) | 103.80(8) |
| O(2)-S(1)-O(1) | 106.98(5) | C(10)-C(8)-C(9) | 107.99(10) |
| O(3)-S(1)-C(1) | 103.46(6) | C(10)-C(8)-C(5) | 113.02(9) |
| C(15)-N(1)-C(11) | 111.92(8) | C(9)-C(8)-C(5) | 113.94(10) |
| C(2)-C(1)-S(1) | 119.04(7) | C(10)-C(8)-C(2) | 114.79(8) |
| C(1)-C(2)-C(1) | 110.87(8) | C(9)-C(8)-C(2) | 113.02(9) |
| C(1)-C(2)-C(7) | 119.03(8) | C(5)-C(8)-C(2) | 93.76(8) |
| C(3)-C(2)-C(7) | 102.20(8) | N(1)-C(11)-C(12) | 108.10(9) |
| C(1)-C(2)-C(8) | 119.24(8) | N(1)-C(11)-C(16) | 111.38(9) |
| C(3)-C(2)-C(8) | 100.45(8) | C(12)-C(11)-C(16) | 112.09(10) |
| C(7)-C(2)-C(8) | 102.27(8) | C(11)-C(12)-C(13) | 111.83(10) |
| O(4)-C(3)-C(2) | 126.50(11) | C(14)-C(13)-C(12) | 110.90(11) |
| O(4)-C(3)-C(2) | 126.29(11) | C(13)-C(14)-C(15) | 110.55(10) |
| C(4)-C(3)-C(2) | 107.21(9) | N(1)-C(15)-C(14) | 109.71(10) |
| C(3)-C(4)-C(5) | 101.19(9) | C(17)-C(16)-C(11) | 115.38(10) |
| C(6)-C(5)-C(4) | 106.16(10) | O(5)-C(17)-C(16) | 111.81(11) |

TABLE 3
Torsion angles [°] for 2-(S)-(2-hydroxyethyl)piperidine camphorsulfonate

| | | | |
|---|---|---|---|
| O(1)-S(1)-C(1)-C(2) | −62.53(10) | C(6)-C(5)-C(8)-C(9) | 172.04(10) |
| O(2)-S(1)-C(1)-C(2) | 59.67(10) | C(4)-C(5)-C(8)-C(9) | 61.78(12) |
| O(1)-S(1)-C(1)-C(2) | 178.29(9) | C(6)-C(5)-C(8)-C(2) | 54.78(10) |
| S(1)-C(1)-C(2)-C(3) | −138.70(8) | C(4)-C(5)-C(8)-C(2) | −55.48(10) |
| S(1)-C(1)-C(2)-C(7) | −20.66(13) | C(1)-C(2)-C(8)-C(10) | −68.98(12) |
| S(1)-C(1)-C(2)-C(8) | 105.45(10) | C(3)-C(2)-C(8)-C(10) | 169.78(9) |
| C(1)-C(2)-C(3)-O(4) | 21.07(16) | C(7)-C(2)-C(8)-C(10) | 64.72(11) |
| C(7)-C(2)-C(3)-O(4) | −106.79(13) | C(1)-C(2)-C(8)-C(9) | 55.48(13) |
| C(8)-C(2)-C(3)-C(4) | 148.08(13) | C(3)-C(2)-C(8)-C(9) | −65.76(11) |
| C(1)-C(2)-C(3)-C(4) | −159.91(9) | C(7)-C(2)-C(8)-C(9) | −170.82(10) |
| C(7)-C(2)-C(3)-C(4) | 72.23(10) | C(1)-C(2)-C(8)-C(6) | 173.50(9) |
| C(8)-C(2)-C(3)-C(4) | −32.90(11) | C(3)-C(2)-C(8)-C(5) | 52.26(9) |
| C(4)-C(3)-C(4)-C(5) | 171.24(13) | C(7)-C(2)-C(8)-C(5) | −52.80(9) |
| C(2)-C(3)-C(4)-C(5) | −1.78(12) | C(15)-N(1)-C(11)-C(12) | 60.30(11) |
| C(3)-C(4)-C(5)-C(6) | −71.31(11) | C(15)-N(1)-C(11)- | −176.14(9) |
| C(3)-C(4)-C(5)-C(8) | 36.49(11) | N(1)-C(11)-C(12)-C(13) | −56.57(13) |
| C(4)-C(5)-C(6)-C(7) | 71.54(11) | C(16)-C(11)-C(12)-C(13) | −179.70(10) |
| C(8)-C(5)-C(6)-C(7) | −36.40(12) | C(11)-C(12)-C(13)-C(14) | 54.84(15) |
| C(5)-C(6)-C(7)-C(2) | 1.97(12) | C(12)-C(13)-C(14)-C(15) | −54.06(16) |
| C(1)-C(2)-C(7)-C(6) | 166.57(10) | C(11)-N(1)-C(15)-C(14) | −61.19(11) |
| C(2)-C(2)-C(7)-C(6) | 70.94(10) | C(13)-C(14)-C(15)-N(1) | 56.87(14) |
| C(8)-C(2)-C(7)-C(6) | 32.74(11) | N(1)-C(11)-C(16)-C(17) | 62.39(13) |
| C(6)-C(5)-C(8)-C(10) | 64.20(12) | C(12)-C(11)-C(16)-C(17) | 176.54(10) |
| C(4)-C(5)-C(8)-C(10) | −174.46(9) | C(11)-C(16)-C(17)-O(5) | 55.73(14) |

FIG. 1: Ortep plot (50%) of 2-(S)-(2-hydroxyethyl)piperidinium α-camphorsulfonate with numbering of the non-hydrogen atoms

1. An insect and mite repellent, characterized by a content of at least one substituted enantiomerically enriched α,ω-amino alcohol derivative of the formula (1) 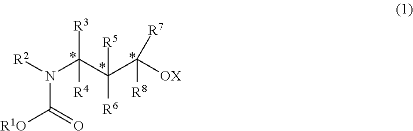
in which
X is hydrogen, COR¹¹ or R¹³, where R¹³ is C₁-C₆-alkyl,
R¹ is C₁-C₇-alkyl, C₃-C₇-alkenyl or C₂-C₇-alkynyl,
R², R¹¹, R¹³ are the same or different and are each C₁-C₆-alkyl or C₂-C₇-alkenyl,
R³ to R⁸ are the same or different and are each hydrogen or C₁-C₆-alkyl, where R² and R³ or R³ and R⁷ or R³ and R⁵ or R⁵ and R⁷, together with the atoms to which they are bonded, may also form a 5- or 6-membered monocyclic ring.

2. An enantiomerically enriched substituted α,ω-amino alcohol derivative of the formula (7) 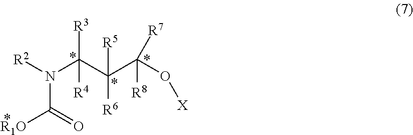
in which
X is hydrogen, COR¹¹ or R¹³, where R¹³ is C₁-C₆-alkyl,
R¹ is C₁-C₇-alkyl, C₃-C₇-alkenyl or C₂-C₇-alkynyl,
R², R¹¹, R¹³ are the same or different and are each C₁-C₆-alkyl or C₂-C₇-alkenyl,
R³ to R⁸ are the same or different and are each hydrogen or C₁-C₆-alkyl, where R² and R³ or R³ and R⁷ or R³ and R⁵ or R⁵ and R⁷, likewise together with the atoms to which they are bonded, may also form a 5- or 6-membered monocyclic ring,
excluding the following substituent combinations a) to f):
a) X=hydrogen, R²=methyl and R¹=tert-butyl
b) X=hydrogen, R¹=ethyl, R⁵=ethyl, R⁶=ethyl
c) X, R³, R⁴, R⁷, R⁸=hydrogen, R¹, R⁵, R⁶=ethyl, R²=methyl
d) X, R³, R⁴, R⁸=hydrogen, R¹, R², R⁵, R⁶=ethyl, R⁷=methyl
e) X, R³, R⁴, R⁷, R⁸=hydrogen, R¹, R², R⁵, R⁶=ethyl
f) X, R³, R⁴, R⁷, R⁸=hydrogen, R¹, R⁵, R⁶=ethyl, R²=n-propyl.

3. An enantiomerically enriched substituted α,ω-amino alcohol derivative of the formula (7) as claimed in claim 2
in which
X is hydrogen or R¹³, where R¹³ is C₁-C₆-alkyl,
R¹ is C₁-C₇-alkyl or C₃-C₇-alkenyl,
R⁴ to R⁸ are the same or different and are each hydrogen or C₁-C₆-alkyl,
R² and R³ together with the atoms to which they are bonded, may also form a 5- or 6-membered monocyclic ring.

4. An enantiomerically enriched substituted α,ω-amino alcohol derivative of the formula (7) as claimed in claim 2
in which
R¹ is C₁-C₇-alkyl, C₃-C₇-alkenyl or C₂-C₇-alkynyl,
X is hydrogen, COR¹¹ or R¹³,
R² and R¹¹ are the same or different and are each C₁-C₆-alkyl,
R³ to R⁸ are the same or different and are each hydrogen or C₁-C₆-alkyl,
R¹³ is C₁-C₆-alkyl,
excluding the following substituent combinations a) to f):
a) X=hydrogen, R²=methyl and R¹=tert-butyl,
b) X=hydrogen, R¹=ethyl, R⁵=ethyl, R⁶=ethyl
c) X, R³, R⁴, R⁷, R⁸=hydrogen, R¹, R⁵, R⁶=ethyl, R²=methyl
d) X, R³, R⁴, R⁸=hydrogen, R¹, R², R⁵, R⁶=ethyl, R⁷=methyl
e) X, R³, R⁴, R⁷, R⁸=hydrogen, R¹, R², R⁵, R⁶=ethyl
f) X, R³, R⁴, R⁷, R⁸=hydrogen, R¹, R⁵, R⁶=ethyl, R²=n-propyl.

5. An enantiomerically enriched substituted α,ω-amino alcohol derivative of the formula (7), characterized in that it is from the group of 1-[(R)-sec-butyloxycarbonyl]-2-(+)-(2-hydroxyethyl)piperidine, 1-[(R)-sec-butyloxycarbonyl]-2-(R)-(+)-(2-hydroxyethyl)piperidine, 1-[(S)-sec-butyloxycarbonyl]-2-(R)-(+)-(2-hydroxyethyl)piperidine or 1-[(R)-sec-butyloxycarbonyl]-2-(R)-(+)-2-hydroxyethyl)piperdine.

6. A process for preparing enantiomerically enriched substituted α,ω-amino alcohol derivatives of the general formula (7) 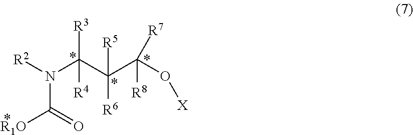
in which
X is hydrogen, COR¹¹ or R¹³, where R¹³ is C₁-C₆-alkyl,
R¹ is C₁-C₇-alkyl, C₃-C₇-alkenyl or C₂-C₇-alkynyl,
R², R¹¹, R¹³ are the same or different and are each C₁-C₆-alkyl or C₂-C₇-alkenyl,
R³ to R⁸ are the same or different and are each hydrogen or C₁-C₆-alkyl, where R² and R³ or R³ and R⁷ or R³ and R⁵ or R⁵ and R⁷, likewise together with the atoms to which they are bonded, may also form a 5- or 6-membered monocyclic ring,
excluding the following substituent combinations a) to f):
a) X=hydrogen, R²=methyl and R¹=tert-butyl
b) X=hydrogen, R¹=ethyl, R⁵=ethyl, R⁶=ethyl
c) X, R³, R⁴, R⁷, R⁸=hydrogen, R¹, R⁵, R⁶=ethyl, R²=methyl
d) X, R³, R⁴, R⁸=hydrogen, R¹, R², R⁵, R⁶=ethyl, R⁷=methyl
e) X, R³, R⁴, R⁷, R⁸=hydrogen, R¹, R², R⁵, R⁶=ethyl
f) X, R³, R⁴, R⁷, R⁸=hydrogen, R¹, R⁵, R⁶=ethyl, R²=n-propyl,
characterized in that
a) enantiomerically enriched am-amino alcohol derivatives of the formula (8) 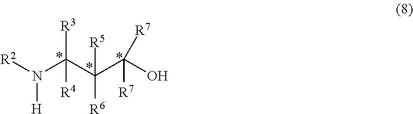
in which
R² to R⁸ are each defined as specified in formula (7) are reacted with chiral carbonic acid derivatives of the formula (9) 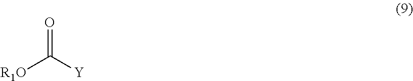
where
R¹ is as defined in formula (7) and
Y is halogen or a leaving group customary in amidation reactions,
optionally in the presence of a diluent and optionally with addition of a base, the resulting compounds of the formula (7) in which X is hydrogen are optionally isolated and are optionally reacted, to obtain compounds of the formula (7) in which X is COR¹¹, with carbonyl chlorides of the formula (4)
R¹¹—COCl (4)
or optionally, to obtain compounds of the formula (7) in which X is R¹³, with alkyl halides of the formula (6)
R¹³—Y (6)
in which
Y is chlorine, bromine or iodine,
b) or chiral α,ω-amino alcohol derivatives or chiral α,ω-amino ethers of the formula (12) 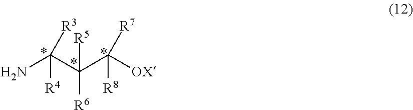
in which
R³ to R⁸ are each defined as specified in formula (7), and in which X′ is hydrogen or R¹³, where R¹³ is C₁-C₆-alkyl, are first reacted with chiral chlorocarbonic esters of the formula (9) 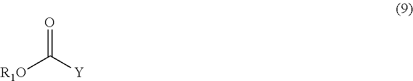
in which
R¹ is a chiral radical defined as specified in formula (7),
optionally in the presence of an acid acceptor and optionally using a diluent, then, in a second reaction step, optionally after isolating the intermediate with a free OH group (X′═H), to prepare compounds of the formula (7) where X═COR¹¹ where R¹¹ is as defined above, with carbonyl chlorides of the formula (4)
R¹¹COCl (4)
and, after optional isolation of the intermediate with a free NH group, also with a compound of the formula (11)
R²—Y′ (12)
in which R² is defined as specified above and Y′ is chlorine, bromine or iodine, optionally in the presence of a base and optionally using a diluent.

7. A method for controlling insects and mites, characterized in that enantiomerically enriched substituted α,ω-amino alcohol derivatives of the formula (1) or (7) are allowed to act on insects and/or mites and/or their habitat.

8. The use of enantiomerically enriched substituted α,ω-amino alcohol derivatives of the formula (1) or (7) for control of insects and/or mites.

9. A process for producing insect and mite repellents, characterized in that enantiomerically enriched substituted α,ω-amino alcohol derivatives of the formula (1) or (7) are mixed with extenders and/or surfactants.
